(19) United States
(12) Patent Application Publication
(10) Pub. No.: US 2014/0065704 A1
(43) Pub. Date: Mar. 6, 2014

(54) CELL COLLECTION SYSTEM

(71) Applicants: Masataka Shirai, Tokyo (JP); Hiroyuki Tsunoda, Tokyo (JP); Hiroko Matsunaga, Tokyo (JP); Kenko Uchida, Tokyo (JP)

(72) Inventors: Masataka Shirai, Tokyo (JP); Hiroyuki Tsunoda, Tokyo (JP); Hiroko Matsunaga, Tokyo (JP); Kenko Uchida, Tokyo (JP)

(21) Appl. No.: 14/111,036

(22) PCT Filed: Apr. 10, 2012
(86) PCT No.: PCT/JP2012/059741

§ 371 Date: Nov. 7, 2013

(30) Foreign Application Priority Data
Apr. 11, 2011 (JP) 2011-087235

(51) Int. Cl. C12M 1/26

(52) U.S. Cl. CPC C12M 33/04
USPC 435/288.7; 435/308.1

(57) ABSTRACT

The present invention provides a simple and highly reliable cell collection system with high throughput and improved in cell collection efficiency. In the present invention, one or more pores are formed in a cell collection plate. One surface of the plate can be directly introduced in e.g., a petri dish, so as to be in contact with a solution containing cells. In this case, means for obtaining an optical image of collected cells from its rear surface is provided to improve reliability and convenience during a cell collection process. Alternatively, the vicinities of the pores in the cell collection plate are only hydrophilized and the other region is made water repellent to improve the efficiency of cell collection.

### TECHNICAL FIELD

The present invention relates to a system for collecting cells separated from tissues such as cultured cells and blood cells for a purpose, such as for analysis, and introducing just a desired number of cells in a reaction plate such as a 96-well plate and a 384-well plate.

### BACKGROUND ART

Recently, gene expression analysis of a single isolated cell such as cultured cells has been conducted (Non Patent Literature 1). In this research, it is necessary to introduce cells into reaction vessels one by one to implement an analysis protocol. Cells are collected by use of a tapered pipette such as a glass capillary and dispensed to plastic reaction vessels one by one. Because of this procedure, it takes time to collect and dispense cells. In the meantime, there is a method of effectively collecting a large number of cells per unit time. This is a method employing a cell sorter using flow-cytometry (Non Patent Literature 2).

Furthermore, to improve throughput of cell collection, the following configuration is known (Patent Literatures 1 and 2): cells are trapped by pores smaller than the cells and formed in a plate. The pores of the plate trapping cells are arranged to face reaction vessels arrayed in a plate one for one by a moving system of a plate. In this manner, cells are put in reaction vessels.

### CITATION LIST

### Patent Literature

Patent Literature 1: U.S. Pat. No. 4,895,805

Patent Literature 2: JP Patent Publication (Kokai) No. 64-80278 (1989)

### Non Patent Literature

Non Patent Literature 1: Wang, D. and Bodovitz, S., Trends in Biotechnology Vol. 28, No. 6, pp. 281-290, 2010

Non Patent Literature 2: Daveley, H. M. and Kell, D. B., Microbiological Reviews, Vol. 60, No. 4, pp. 641-696, 1996

### SUMMARY OF INVENTION

### Technical Problem

In place of suctioning cells one by one by a pipette, it is necessary to develop a system of simply trapping cells and ejecting them into reaction vessels.

In a method of using a flow-cytometer (for example, Non Patent Literature 2), a flow-cytometer has a problem since it is extremely expensive due to its complicated liquid feeding system. Furthermore, a microscopic image of the cell to be dispensed cannot be checked in advance. Moreover, since impulsive force such as an electric field and ultrasonic wave is directly applied to cells in order to separate only a desired cell, the cells are often damaged.

To solve these problems, a method of forming pores smaller than the diameter of cells for collecting cells and trapping cells contained in a solution by suctioning the solution is known (Patent Literatures 1 and 2). This method has high throughput since cells are collected simultaneously and, in addition, damage to cells can be reduced by appropriately controlling the pressure for suctioning the solution. However, to biologically analyze the collected cells by use of a reagent such as an enzyme (for example, a quantitative analysis for evaluating nucleic acids and proteins), it is effective to dispense cells to wells of a 96-well plate or a 384-well plate (hereinafter, generally referred to as a “reaction-vessel plate”). This is because various analyzers and reagent dispensing machines are formed so as to fit to the intervals between reaction vessels and the shapes of the reaction vessels and thus, these analyzers and machines can be directly used. Since reaction vessels herein are arrayed in a 96-well plate at intervals of e.g., 9 mm, it is necessary to form cell collection pores in a cell collection plate so as to correspond to the intervals to trap cells at the same intervals. However, since the area of the region not involved in trapping cells is larger than the area of cell collection pores, cells are adsorbed to the region, with the results that cells are broken and destroyed. Furthermore, when cells are introduced in a reaction vessel plate, cells are inserted between the reaction vessel plate and a cell collection plate to prevent alignment of both plates. Such an incident is a matter of concern.

Furthermore, after examining if a cell was successfully trapped by a cell collection pore; or two or more cells were accidentally trapped; and further examining the shape and type of the trapped cell under microscopic observation, introducing cells into reaction vessels is effective. However, for examination, it is necessary to obtain a microscopic image always focused on the cell collection plate. In collecting cells suspending in e.g., a petri dish, while a stereoscopic microscope is focused on a certain position within the petri dish, if an attempt is made to collect cells by moving a cell collection plate without limit in a solution containing cells, an image of a region in the vicinity of a cell collection surface cannot be obtained.

Accordingly, an object of the invention is to provide a means for simply introducing cells one by one into wells in a 96-well plate or a 384-well plate generally used in medical care and biological research institutions by a mechanically inexpensive apparatus, with high throughput.

### Solution to Problem

The present inventors intensively studied with a view to solving the aforementioned problems. As a result, they found that if a means for obtaining an optical image of vicinities of pores for trapping cells is integrated with a system for trapping and ejecting cells, the trapping state of the cell can be observed and cells can be simply and reliably trapped and ejected. They further found that if the vicinities of the pores for trapping cells are hydrophilized and the other region is made water repellent, adsorption of unnecessary cells is prevented and cell collection efficiency can be improved. Based on these findings, the present invention was accomplished. In short, the present invention is as follows.

[1] A cell collection system, having

a cell collection plate having at least one pore formed therein and one surface that can be soaked or in contact with a solution comprising a cell,

means for trapping a cell in the pore by suctioning the solution comprising a cell from the pore,

means for ejecting the cell trapped in the pore, and

means for obtaining an optical image of a vicinity of the pore in the cell collection plate.

[2] The cell collection system according to [1], in which a vicinity of the pore in the surface of the cell collection plate are hydrophilic and/or the other portion except the vicinity of the pore of the surface is water repellent.

[3] A cell collection system, having

a cell collection plate having at least one pore formed therein and one surface that can be soaked or in contact with a solution comprising a cell,

means for trapping a cell in the pore by suctioning the solution comprising a cell from the pore, and

means for ejecting the cell trapped in the pore, in which

the vicinity of the pore in the surface of the cell collection plate is hydrophilic and/or the other portion except the vicinity of pore is water repellent.

[4] The cell collection system according to any one of [1] to [3], in which the pore is two-dimensionally or one-dimensionally arrayed in the cell collection plate.

[5] The cell collection system according to any one of [1] to [4], in which the diameter of the pore is smaller than the diameter of the cell.

[6] The cell collection system according to any one of [1] to [5], in which at least a part of the cell collection plate is transparent.

[7] The cell collection system according to any one of [1] to [6], in which the periphery of the pore in the cell collection plate is transparent.

[8] The cell collection system according to any one of [1], [2] and [4] to [7], in which the means for obtaining an optical image of the vicinity of the pore in the cell collection plate comprises a light fiber bundle.

[9] The cell collection system according to any one of [1] to [8], in which the cell collection plate in the vicinity of the pore has a shape projecting on the side of the surface to be soaked or in contact with the solution comprising a cell.

[10] The cell collection system according to any one of [1] to [9], further comprising illumination means.

[11] The cell collection system according to any one of [1], [2] and [4] to [10], further comprising a computer and software for analyzing the optical image of the vicinity of the pore, wherein the cell collection system automatically recognizes the trapping of the cell in the pore based on contrast difference between an image of the pore and the image of the other portion except the pore.

[12] The cell collection system according to any one of [1], [2] and [4] to [11], in which the means for obtaining an optical image comprises a fluorescence excitation light source, an optical system for detecting fluorescence and an imaging device for obtaining the fluorescent image.

[13] The cell collection system according to any one of [1] to [12], further comprising a mechanism for washing the cell collection plate.

[14] A cell collection and dispensing system comprising

the cell collection system according to any one of [1] to [13], and

a reaction vessel plate having at least one reaction vessel to which a cell is to be dispensed,

in which the pore is formed in the cell collection plate at an interval corresponding to an array interval of the reaction vessel in the reaction vessel plate.

[15] The cell collection and dispensing system according to [14], in which the number of the pore in the cell collection plate matches with the number of the reaction vessel in the reaction vessel plate.

[16] The cell collection and dispensing system according to [14] or [15], in which the cell collection plate and the reaction vessel plate have means for aligning the cell collection plate to the reaction vessel plate.

[17] The cell collection and dispensing system according to [16], in which the alignment means is provided such that the pore in the cell collection plate is positioned off the center of the corresponding reaction vessel of the reaction vessel plate.

[18] The cell collection and dispensing system according to any one of [14] to [17], in which the cell collection plate is discretely provided from the solution comprising a cell and the reaction vessel plate and can be moved without limit.

[19] The cell collection and dispensing system according to any one of [14] to [18], further comprising a means capable of taking the optical image focused within the reaction vessel when the cell trapped in the pore of the cell collection plate is dispensed to the reaction vessel plate.

### Advantageous Effects of Invention

A cell collection system and a cell collection and dispensing system are provided by the present invention. The systems according to the present invention are capable of simply isolating cells, collecting them and dispensing the collected cells to reaction vessels in an existing reaction vessel plate. According to the present invention, the reliability of cell collection can be improved by a simple and small system. In addition, the efficiency of cell collection can be improved.

Problems, configurations and effects other than the aforementioned ones will be clearly understood by the descriptions of the following embodiments.

### BRIEF DESCRIPTION OF DRAWINGS

FIG. 1 shows an example of the structure of the system of the present invention in a step of collecting cells according to Example 1.

FIG. 2 shows a surface treatment pattern of the cell collection surface of Example 1.

FIG. 3 shows an example of the structure of the system of the present invention in a step of dispensing cells according to Example 1.

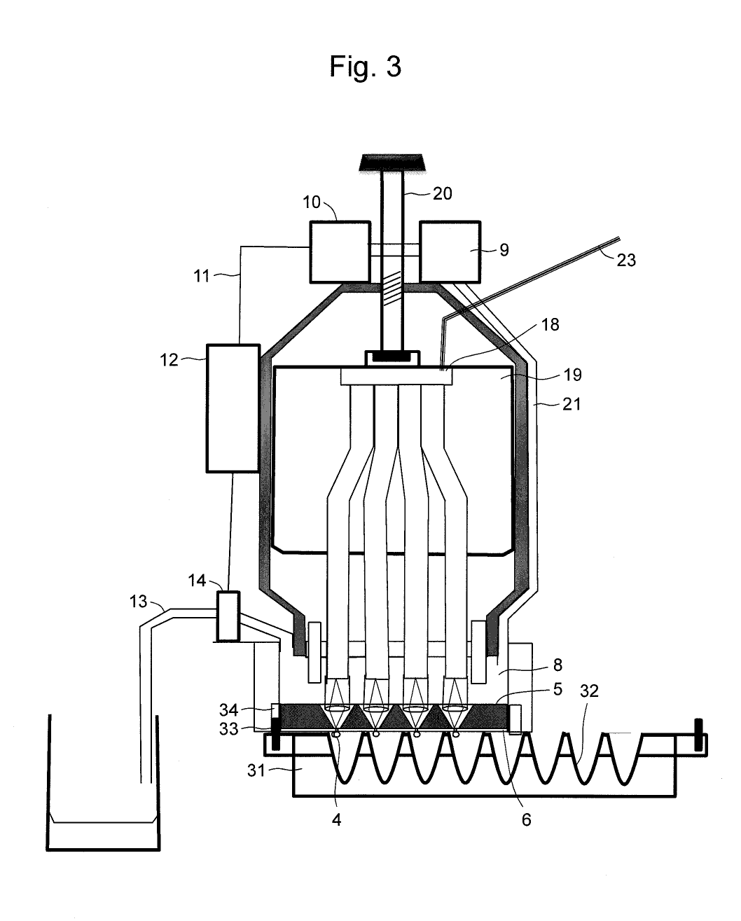

FIG. 4 shows a sectional shape of the cell collection surface of Example 1.

FIG. 5 shows an example of the structure of the system of the present invention in a step of collecting cells according to Example 2.

FIG. 6 shows an example of the structure of the system of the present invention in a step of collecting cells according to Example 3.

FIG. 7 shows surface treatment pattern of the cell collection surface of Example 3.

FIG. 8 shows the shape of liquid drop on a portion (A) hydrophilized and the shape of a liquid drop on a portion (B) of water repellant.

### DESCRIPTION OF EMBODIMENTS

Now, the present invention will be more specifically described below. The present application claims the priority right of JP Patent Application No. 2011-087235 filed on Apr. 11, 2011, and incorporates herein by reference the contents described in the specification and/or the drawings of the patent application.

The present invention relates to a cell collection system for trapping cells from a solution containing the cells and ejecting the cells in desired sites. To describe more specifically, a cell collection plate is directly introduced in a petri dish or a flask generally used for cell culture. Only one surface (collection surface) of the plate is allowed to contact with a solution suspending cells. The solution is suctioned from the other surface (rear surface) to trap the cells. The cell collection plate is designed to be movable without limit Particularly, the cell collection plate is desirably small and light so as to manually move it. In this case, an imaging device and optical system for forming an image are integrated with the cell collection plate in order to observe images of cells from the rear surface of the cell collection plate. In this manner, a focus position would not move even if the cell collection plate is moved. Alternatively, or additionally, the vicinities of the pores in the cell collection surface of the cell collection plate are hydrophilized and the other portion is made water repellent to prevent adsorption of cells to the portions except the pores.

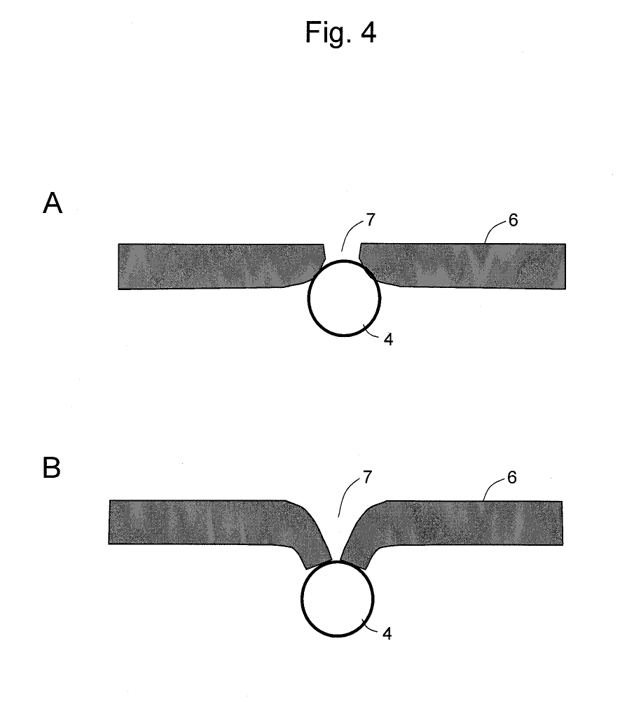

Accordingly, the cell collection system according to the present invention has a cell collection plate having at least one pore formed therein and having one surface that can be soaked or in contact with a solution containing cells. The cell collection plate may have any size and shape and may be formed of any material as long as it has a pore. Preferably, the cell collection plate has a size and shape suitable for the size and shape of a petri dish to which a solution containing cells is introduced and the size and shape of a reaction vessel plate having vessels to which cells are to be dispensed. For example, a circular, square, and rectangular flat-plate can be employed. Herein, it is preferable that the pore portions of the cell collection plate project on the side of the surface (cell collection surface) which is to be soaked or in contact with a solution containing cells (for example, FIG. 4B).

The size of the cell collection plate can be 3×3 mm to 500×500 mm and preferably 10×10 mm to 85×125 mm. Furthermore, the thickness can be 0.1 mm to 10 mm and preferably 0.3 to 5 mm.

Examples of the material for the cell collection plate include, but are not limited to, resins (for example, a polyester resin, polystyrene, a polyethylene resin, a polypropylene resin, an ABS resin (Acrylonitrile Butadiene Styrene resin), nylon, an acrylic resin, a fluorine resin, a polycarbonate resin, a polyolefin resin, a polyurethane resin, a polyvinylidene chloride, a methylpentene resin, a phenolic resin, a melamine resin, a peek resin, an epoxy resin and a vinyl chloride resin), metals (for example, gold, silver, copper, aluminum, tungsten, molybdenum, chromium, platinum, titanium, nickel), alloys (for example, stainless steel, hastelloy, inconel, monel metal, duralumin), glass (for example, glass, quartz glass, fused quartz, synthetic quarts, alumina, sapphire, ceramics, forsterite and photosensitive glass), semiconductor materials, silicon and rubbers (for example, natural and synthetic rubbers). A plurality of materials may be used in combination. For example, the main portion of a cell collection plate and a portion in contact with a cell collection surface may be formed of different materials. To describe more specifically, the main portion of a cell collection plate is formed of a rigid material in order to keep sufficient strength; whereas the portion in contact with a cell collection surface is formed of a transparent material in order to observe an optical system as described later.

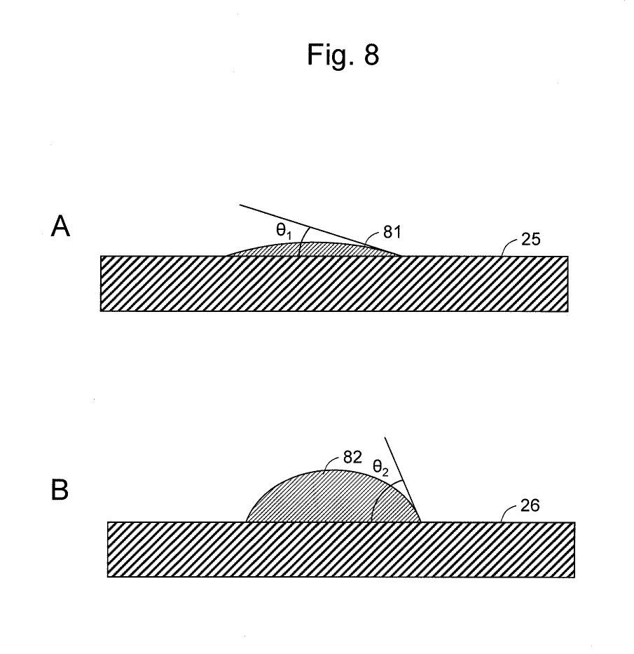

The vicinities of the pores in the cell collection surface of a cell collection plate are preferably hydrophilic. The term “vicinity of a pore” refers to the peripheral region of the pore including the pore. A region of at least 10 μm around a pore, preferably at least 30 μm, and at most 2 mm, preferably at most 1 mm can be mentioned. For example, a region around a pore having a diameter of 10 to 200 μm, preferably 30 to 100 μm, more specifically, 50 μm, is hydrophilized. As the hydrophilization method, a method known in the art can be used. Examples thereof include a UV ozone treatment method, which is a method of applying UV light of 254 nm or 176 nm in wavelength under an oxygen atmosphere, and a method, in which resist patterning is performed and thereafter a silane coupling agent having an OH group as a functional group is reacted only with an opening portion to form a hydrophilic pattern. Owing to the hydrophilic treatment, liquid drops of a solution containing cells can be easily adsorbed to hydrophilic portions, i.e., the vicinities of the pores. In addition to this or independently, the portion of the cell collection surface of a cell collection plate except the vicinities of the pores is preferably water repellent. Water repellency is obtained by applying a water repellent treatment to the portion except the pores or preparing a cell collection plate from a water repellent material (for example, polyvinylidene chloride). Owing to the water repellency, liquid drops of a solution containing cells are prevented from adsorbing to the portion except the vicinities of the pores. Note that, in the present invention, “hydrophilicity” and “water repellency” are defined by a water contact angle of a liquid drop with respect to a hydrophilic and water repellent surfaces, respectively. The water contact angle of a liquid drop with respect to a hydrophilic surface is smaller than the water contact angle of a liquid drop with respect to a water repellent surface. The “hydrophilicity” and “water repellency” are relatively defined by these two angles (for example, see FIG. 8).

Furthermore, it is preferable that at least a part of a cell collection plate is transparent. For example, the peripheral portions of pores in a cell collection plate can be transparent. The term “peripheral portions of pores” refers to the peripheral regions of pores including the pore. A region of at least 10 μm around a pore, preferably at least 30 μm, and at most 2 mm, preferably at most 1 mm can be mentioned. Alternatively, the whole cell collection plate may be formed of a transparent material. Owing to this, an optical image of cells can be easily observed by an optical system (described later).

In the cell collection plate, at least one pore is formed. The diameter of the pore is set to be smaller than the diameter of the cell to be collected. For example, the size of prokaryotic cells is about 1 to 10 μm; whereas the size of eukaryotic cells is about 5 to 100 μm. The size of a pore can be determined based on the size of specific cells to be collected. Specifically, since the size of animal cells is generally 5 to 10 μm, the diameter of pores can be determined to be 2 to 5 μm, for example, 4 μm. Furthermore, the shape of pores, which is not particularly limited, can be e.g., circular, rectangular, square, rectangular and triangular shapes. The shape of pores in the thickness direction of a cell collection plate, which is not particularly limited, can be e.g., a taper shape and a cylindrical shape. Furthermore, the number of pores, which is not particularly limited, can be e.g., 1 to 1000, for example, 1, 4, 16, 96 and 384.

Pores are preferably two-dimensionally or one-dimensionally arrayed in a cell collection plate. For example, pores can be one-dimensionally (e.g., four pores) or two-dimensionally arrayed (4×4 pores). It is preferable that the intervals of pores to be arrayed match with the array intervals of reaction vessels in a reaction vessel plate to which cells are to be ejected. For example, pores can be arrayed at intervals of 5 to 50 mm. The number of pores in a cell collection plate may be the same or different from the number of reaction vessels in a reaction vessel plate. For example, when a 96-well plate is used as a reaction vessel plate, a cell collection plate having 96 pores arrayed at the same intervals as those of reaction vessels can be used or a cell collection plate having 16 pores arrayed at the same intervals as those of reaction vessels can be used.

A method for forming pores in a cell collection plate may be selected from the methods known in the art depending upon the type of material to be used as a cell collection plate and the size of pores. For example, cutting processing, punching processing and excimer laser processing can be appropriately selected.

Furthermore, the cell collection system of the present invention has a means for trapping cells in pores by suctioning a solution containing cells through the pores of a cell collection plate and a means for ejecting the cells trapped in the pores. For example, a means for applying pressure difference between the cell collection surface of a cell collection plate and the rear surface thereof and a means for taking the pressure difference back to the normal or a means for inversely applying the pressure difference can be used. To describe more specifically, a discharge tube for suctioning a solution containing cells, connected to the rear surface of a cell collection surface is provided at a lower level than the solution containing cells. In this manner, pressure difference can be gravitationally applied between a cell collection surface and its rear surface. Alternatively, pressure difference is gravitationally applied between a cell collection surface and its rear surface by suctioning a solution containing cells from pores by use of a suctioning means such as a pump.

It is preferable that the cell collection system of the present invention has a means for obtaining an optical image of vicinities of pores in a cell collection plate. As such a means, an optical system known in the art can be mentioned. Examples thereof can include a light fiber bundle. Examples of the optical system that can be used include a lens (field lens, an objective lens and an image formation lens), a mirror, a filter, an imaging device (e.g., CMOS sensor, CCD sensor). Furthermore, for example, if the cells to be collected emit fluorescence, a fluorescence excitation light source, an optical system for detecting fluorescence and an imaging device for obtaining fluorescent images can be used as the means. Whether cells were trapped in pores or whether a single cell was successfully trapped in each pore or not, and whether 2 or more cells were trapped can be determined by obtaining an optical image of vicinities of pores. Based on the determination, accuracy and reliability of a reaction later performed can be guaranteed.

It is preferable that the cell collection system of the present invention further has a computer and software for analyzing an optical image of vicinities of pores in a cell collection plate. Owing to this, it is possible to automatically recognize the trapping of a cell in a pore based on contrast difference between an image of the pore and an image of the portion except pores.

It is preferable that the cell collection system of the present invention further has an illumination means. As the illumination means, any type, shape and size of illumination means can be used as long as it is known in the art. Examples thereof include a white light bulb and white LED. The illumination means may be integrated with a cell collection system or detachable, or may be a discrete part.

Furthermore, it is preferable that the cell collection system of the present invention further has a mechanism for washing a cell collection plate. The washing mechanism may be integrated with a cell collection system or a discrete part, which is used by connecting it at the time of washing. In the washing mechanism, a washing liquid inlet pipe and a washing liquid disposable container are included.

The present invention further relates to a cell collection and dispensing system having a cell collection system of the present invention and a reaction vessel plate having at least one reaction vessel to which cells are dispensed. The cell collection and dispensing system of the present invention has a simple configuration as follow: when trapping of cells is completed in the cell collection system, suctioning of a solution is terminated; however, suction force is still maintained to facilitate movement of cells from a petri dish or a flask to a reaction vessel plate.

As described above, in the cell collection and dispensing system of the present invention, pores of a cell collection plate are arrayed at intervals corresponding the intervals between reaction vessels in a reaction vessel plate. Furthermore, the number of pores in the cell collection plate are preferably the same as the number of the reaction vessels of the reaction vessel plate; however the number of pores may differ.

The reaction vessel plate can be a reaction plate known in the field to which the reaction to be carried out pertains. Specifically, the reaction vessel plate preferably has a solid planar surface, which is insoluble in water and does not melt during heat denaturation. Examples of a material for the plate include a metal, alloy, silicon, a glass material and a plastic such as a resin. Furthermore, the shape of the reaction vessel plate is a planar surface having compartmentalized reaction vessels. For example, a titer plate, a porous or pore array is mentioned.

The pores of the cell collection plate are arrayed at the intervals corresponding to those of reaction vessels arrayed in the reaction vessel plate. Therefore, the cells trapped in the cell collection plate can simply correspond to the positions of the reaction vessels one for one. In the cell collection and dispensing system of the present invention, it is preferable that the cell collection plate and the reaction vessel plate have means for aligning the cell collection plate with the reaction vessel plate. The aligning means may have a snap-fit system. For example, a pin-and-hole snap-fit system, a projection-depression snap-fit system can be mentioned. It is effective to fix such aligning means to a reaction vessel plate and a cell collection plate or the periphery thereof. It is preferable that the aligning means are provided to attain the state where the pores (i.e., trapped cells) of the cell collection plate are positioned off the center of a reaction vessel of the reaction vessel plate. Particularly, it is preferable that the aligning means is provided to attain the state where pores are positioned near the wall of a reaction vessel such that a solution containing cells reaches near the bottom of a reaction vessel along the wall. Owing to this, damage to cells can be effectively reduced.

Furthermore, in the cell collection and dispensing system of the present invention, it is preferable that the cell collection plate is discretely provided from a solution containing cells and a reaction vessel plate and movable without limit.

It is preferable that the cell collection and dispensing system of the present invention further has a means for obtaining an optical image by focusing an optical system on a point within a reaction vessel when the cells trapped in the pores of the cell collection plate are dispensed to the reaction vessel plate. For example, as such a means, the aforementioned means for obtaining an optical image of the regions in the vicinity of pores may be used.

The cell collection system and the cell collection and dispensing system of the present invention are suitable in the case where cells are desired to be dispensed into reaction vessels one by one for culturing or analysis. The cells to be collected and dispensed are not particularly limited as long as they are subjected to culture or an analytic reaction. Prokaryotic cells and eukaryotic cells (particularly, animal cells) can be used. The solution containing cells can be appropriately selected as long as it is a solution suitable for target cells. A buffer controlled in isotonicity (for example, phosphate buffered saline), a culture medium or the like can be used. The density of the cells can be appropriately selected depending upon the number of cells to be collected and the number of pores in a cell collection plate.

Using the cell collection system and cell collection and dispensing system of the present invention, a cell collection plate is directly inserted into a petri dish or a flask generally used in culturing cells; only one surface (collection surface) of the cell collection plate is allowed to be in contact with a solution suspending cells and the solution containing cells is suctioned from the other surface (rear surface) to trap cells. At the time of completion of alignment between the cell collection plate and the reaction vessel plate, the pressure is inversely applied to the pores for collecting cells to eject cells together with the solution. In this manner, cells are dispensed to reaction vessels. Since cells are collected together with a solution herein, damage to cells can be reduced.

As described above, owing to the present invention, a cell collection system and a cell collection and dispensing system are provided. In the systems according to the present invention, cells are easily isolated, collected and dispensed to an existing reaction vessel plate. Particularly, by taking an optical image of cells simultaneously with collection of the cells, a reaction vessel which fails to catch a cell and a reaction vessel to which two or more cells are dispensed can be distinguished. Furthermore, whether a predetermined type of cell can be caught or not is confirmed and then subjected to the following analysis. In short, reliability in collecting cells by a simple and small system can be improved. Furthermore, since only the regions in the vicinity of pores in a cell collection plate are hydrophilized, and the other region remains water repellent, the efficiency of cell collection can be improved.

### EXAMPLES

Now, referring to the drawings, specific examples of embodiments of the present invention will be described below. However, these Examples are just examples for realizing the present invention and should not be construed as limiting the present invention.

### Example 1

This Example is a basic embodiment of the present invention. The example shows a system of simultaneously trapping 16 cells (particularly, animal cells) cultured and suspended in a petri dish having a diameter of 50 mmφ or more, and ejecting them to a 96-well plate.

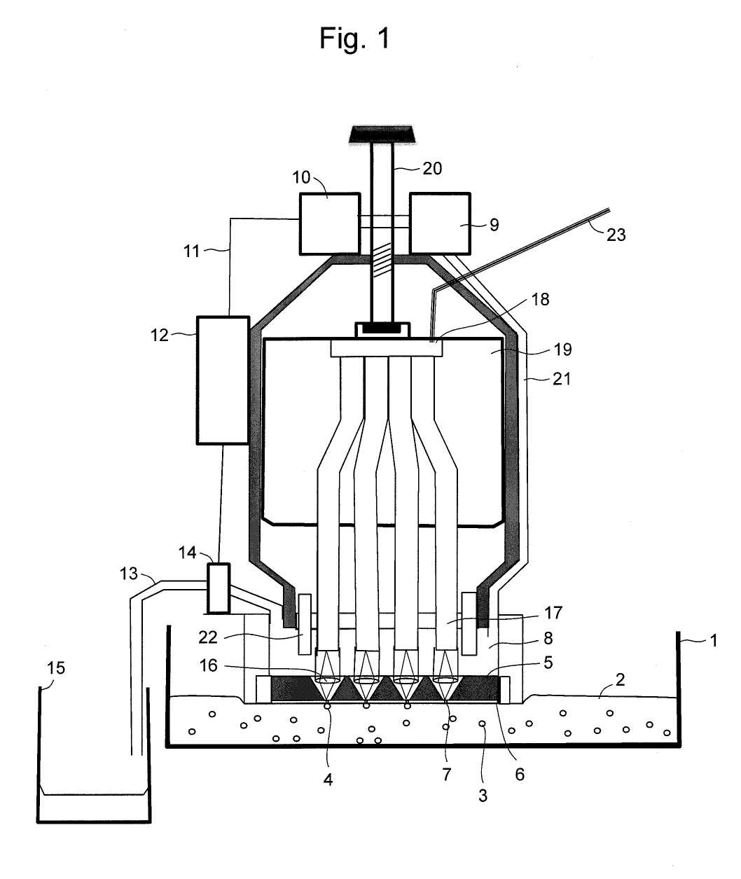

FIG. 1 shows the structure of the system in a step of trapping cells suspending in a petri dish. In a petri dish 1 made of glass and having a diameter of 60 mm, a PBS buffer 2 was introduced to suspend cultured cells 3. The density of the cells is controlled to be about 1000 cells/mL. A cell collection system (all structural components except the petri dish 1 and the PBS buffer 2) is soaked in the solution such that one surface is allowed to be contact with the solution. To the leading-edge of the cell collection system, a cell collection plate 5 is provided and held such that one surface (cell collection surface) comes to be in contact with the PBS buffer 2 containing cells 3. In the cell collection plate 5, pores 7 for collecting cells are formed and the diameter of an opening portion in the cell collection surface is set at 4 μm. The pores (16 pores) are arrayed in 4×4 at intervals of 9 mm so as to correspond to the intervals of the reaction vessels arrayed in a 96-well plate. In this Example, the cell collection plate 5 is formed of two layers, a polyvinylidene chloride film of 5 μm in thickness was used in a portion 6 in contact with a cell collection surface. Other compounds such as polypropylene, polycarbonate and a cyclic polyolefin may also be used. Punching processing used herein was an excimer laser processing. The rear surface side of the cell collection plate 5, which plays a role in maintaining the shape, was molded by cutting a peek resin. The pore was designed to have a taper shape having a diameter of 1 mm near the cell collection surface and a diameter of 3 mm in the rear surface. The inner space of a suction chamber 8 is filled with PBS buffer before cells are trapped. This is realized by feeding the solution from a solution reservoir (storing PBS buffer 2) through a liquid-feeding tube 21 by means of a pump 10. Furthermore, feeding (with application of pressure) of the buffer to the suction chamber 8, suctioning (by reducing pressure) and opening to the atmosphere are controlled by sending a control signal to the pump 10 through a signal line 11 by a controller 12. The size of a cell collection plate was set at 45×45 mm to 500×500 mm and the thickness was set at 1 mm

Next, for trapping cells, it is necessary to produce pressure difference between the cell collection surface and rear surface of pores 7 of the cell collection plate 5. For this, a discharge tube 13 was provided to an appropriate position lower than the liquid-surface of the buffer in the petri dish 1 and a flow rate was controlled by a flow controller 14 provided in the middle of the discharge tube 13. As described above, the pressure difference is gravitationally produced. This is because if a high pressure difference is applied to pores 7, cells may be damaged. If small pressure accurately controlled can be applied by a pump, the pump 10 may be used in place of the flow controller 14. Note that, similarly to the pump 10, the flow controller 14 was also controlled by a controller 12. Reference numeral 15 denotes a wastewater container, which collects e.g., discharged PBS, unnecessary small cells passing through pores and trashes.

To confirm a cell 4 trapped by a pore 7 by an optical microscope, a transparent material was used in the portion 6 in contact with the cell collection surface. Thus, the transparent opening portion has a size of 1 mmφ and an optical image of this region can be obtained. To match with the opening portion, an aspheric surface lens 16 and a fiber bundle 17 (fiber core system: 3 μm, a bundle diameter: 1 mm) were used. An image of the cell 4 trapped in the opening portion of the pore 7 is formed on the surface of the fiber bundle 17 through the lens 16 and transmitted to a CMOS sensor 18. If the chip size of the CMOS sensor 18 increases, the cost of the device increases. Thus, as a possible configuration, 16 images were separately obtained by use of fibers and synthesized into one. Reference numeral 22 denotes a white LED for illumination. An image obtained by the CMOS sensor 18 is sent to an external PC and an external display through a signal line 23. To control a focus position, a micrometer 20 is provided, which moves an optical module 19, in which the CMOS sensor 18, fiber bundle 17 and aspheric surface lens 16 are integrated therein, up and down. Owing to this, it is possible to determine which pore traps a cell, which position of a pore traps 2 or more cells, and which position of a pore traps an abnormal shaped cell, and avoid adding an analysis reagent after dispensing cells to reaction vessels to save reagent cost. In addition, correspondence between individual cells analyzed and optical images of the cells can be obtained.

A first purpose of the optical system of the present invention is to determine whether a single cell was trapped in a pore or not and whether two or more cells were trapped in a pore. Because of this, it is not necessary to take a clear optical image of a cell from the rear surface of a pore. In an extreme case, if it is only determined that the contrast of a contour image of a pore trapping a single cell appropriately changes, the purpose of the optical system can be almost attained.

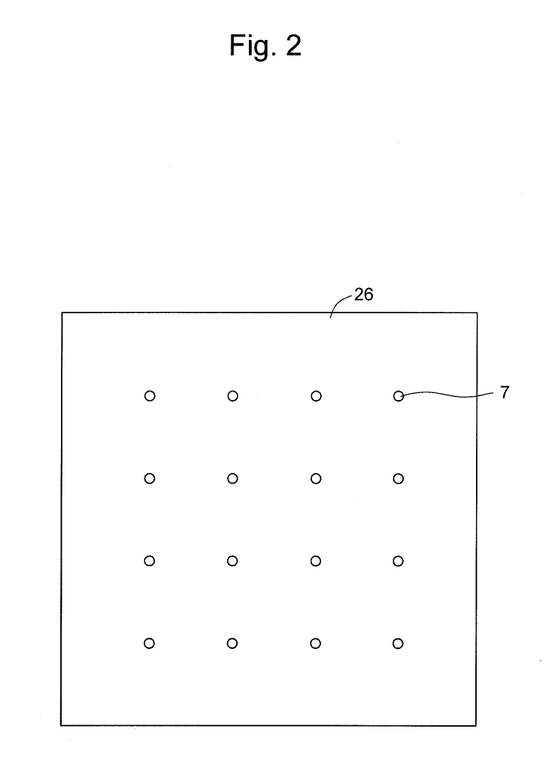

FIG. 2 shows an arrangement pattern of pores 7 in the cell collection surface. Sixteen pores 7 for collecting cells are arrayed at intervals of 9 mm in the form of a square lattice. Furthermore, the region except the pores 7 is a water repellent surface portion 26.

In this case, the pressure of the inner portion of the suction chamber 8 is controlled by the controller 12 so as to keep an appropriate negative pressure (weak pressure enough to keep cells from destroying) relative to the exterior portion.

Subsequently, in order to dispense the trapped cells 4 to reaction vessels 32 of a reaction vessel plate 31 one by one, the cell collection plate 5 (the portion 6 in contact with a cell collection surface in the Example) is allowed to be in close contact with the reaction vessel plate 31. At this time, in order to align the pores 7 with reaction vessels 32, an aligning pin 33 is provided to the reaction vessel plate 31; whereas an aligning hole 34 is formed to the cell collection plate 5. Furthermore, the position of the aligning hole 34 is controlled to attain the state where the position of each of the pores 7 is off the center of a reaction vessel 32 such that, when a solution is ejected, the cell reaches the bottom of the reaction vessel 32 along the wall surface of the reaction vessel 32.

Cells are dispensed by ejecting PBS buffer from the solution reservoir 9 by use of the pump 10. At this time, needless to say, the flow rate is set to be 0 by the flow controller 14.

Furthermore, to confirm whether cells are ejected or not, an optical module 19 can be moved by a micrometer 20 such that a focus position comes to be near the bottom of the reaction vessel 32.

In this Example, whether cells were collected or not is visually confirmed primarily based on a microscopic image. This process can be automatically performed. For automatic operation, an image obtained by the CMOS sensor 18 is transmitted to PC by use of the signal line 23. When a cell is trapped, the refraction index within the pore increases compared to the pore trapping no cell. As a result, the reflection rate of light from white LED 22 for illumination changes and thus the contrast of a ring constituting the contour of the pore changes. If the contrast change is checked by an image recognition software, whether a cell is trapped or not can be automatically determined. In this Example, light is applied from the rear surface side of the cell collection plate 5. However, if light is applied from the cell collection surface side, the same contrast change occurs.

Furthermore, FIG. 4 shows the shape of the portion in the vicinity of a pore 7 in the cell collection surface of the cell collection plate 5. In the Example, two types of shapes were prepared. FIG. 4A shows a shape in which a pore diameter reduced from the cell collection surface side toward the rear surface. Note that the pore diameter is represented by the smallest opening size. In the case of such shape, if a cell is easily deformed by stress, the cell reaches deeply into the pore. Since contact area with a wall surface is large, the cell attaches with the wall surface. In this case, when the solution is allowed to flow in an inverted direction during an ejection process, the cell may be destroyed or remain together with the solution without being ejected. To reduce such a problem, the pore is formed so as to project toward the cell collection surface side as shown in the sectional shape of FIG. 4B. Owing to this design, the contact area with the wall surface is kept constant.

Note that in this Example, sixteen pores 7 were arrayed in the cell collection plate 5. Needless to say, the number of pores may be 1, 96 or 384. In the case of a single cell, the possibility of introducing unnecessary cells into a reaction vessel can be reduced. In the cases of 96 cells and 384 cells, throughput can be improved.

### Example 2

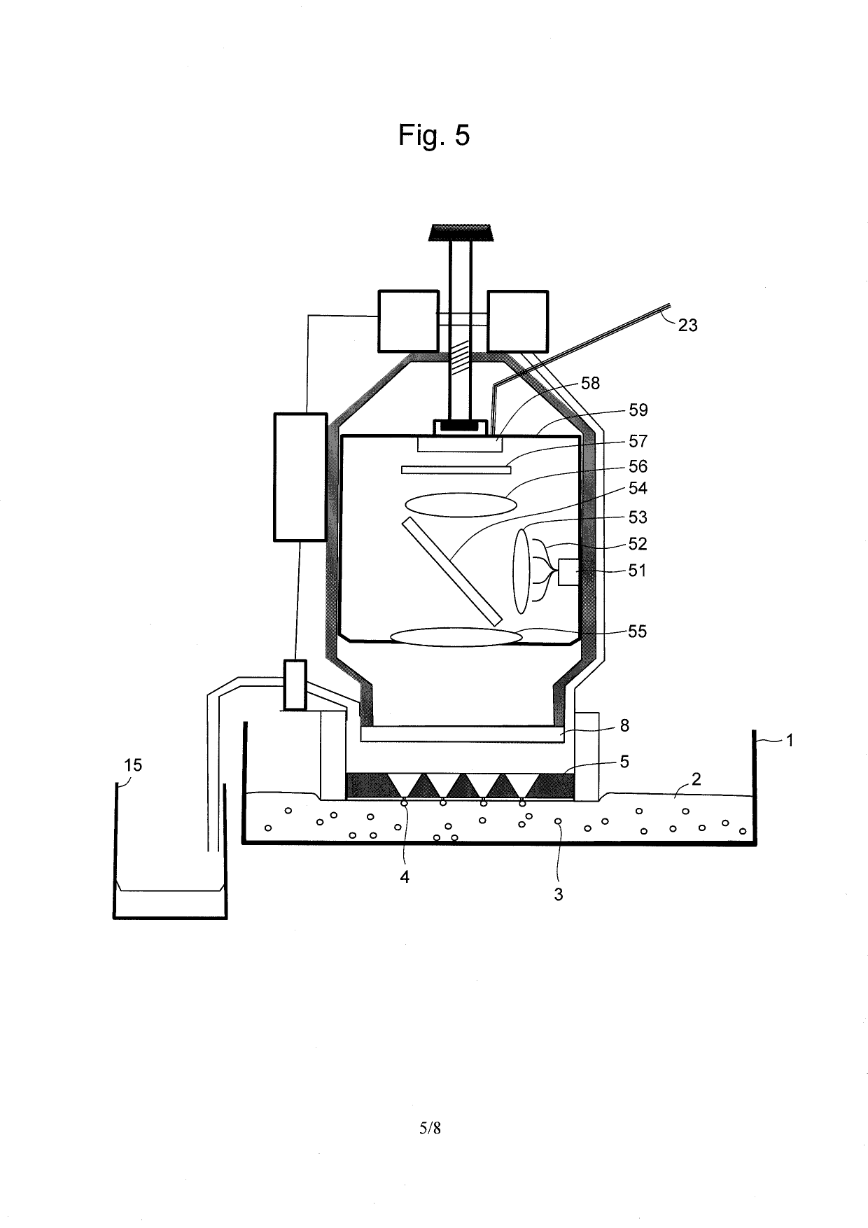

The system of this Example has a function of a fluorescent microscope. A case of introducing only a cell expressing a desired protein to a reaction vessel plate will be described. FIG. 5 shows the structure example of the system in a step of collecting cells of the Example.

In the petri dish 1, only desired cells 3 to be collected are labelled with a green fluorescent protein (GFP). Furthermore, the cells collected are cancer cells, which are larger than other cells (blood cells) and less flexible. Because of the features, in trapping the cells 3 by suctioning a PBS buffer 2 through pores 7, many unnecessary blood cells pass and move toward a suction chamber 8 and are discharged, as wastewater, in a wastewater container 15. In this manner, only desired cells are trapped. At the same time, since the cells are tagged with a fluorescent label, it is possible to detect fluorescence. FIG. 5 shows a configuration for confirming whether trapped cells 4 are desired cells or not by a fluorescent meter. A fiber bundle 52, which fan-outs excitation light having a wavelength of 488 nm to an output portion of a semiconductor laser 51 serving as an excitation light source depending upon the number of cells required to be excited; a field lens 53, which converges the laser beams output from the fiber into the portion in the vicinity of pores 7; a dichroic mirror 54, which reflects excitation light and transmits fluorescence from the fluorescent label (GFP); an objective lens 55, which collects fluorescence; an imaging lens 56, which forms a fluorescent image on an imaging device (cool CCD58); a band-pass filter 57, which removes scattering light from the excitation laser light and Raman scattering from water to reduce background of fluorescence; and a cool CCD58 are disposed in an optical module 59.

In suctioning the buffer to trap the cells 3, almost all unnecessary blood cells pass through the pores 7 and are discharged in a wastewater container 15; whereas, the cells having GFP fixed on the cell surface and emitting fluorescence are only trapped by the cell collection plate 5. Furthermore, whether trapped cells 4 truly emit fluorescence can be confirmed by the optical module 59.

### Example 3

This Example, similarly to Example 1, shows a system for simultaneously trapping a plurality of cells 3 suspending in a petri dish and ejecting them to a 96-well plate. To avoid deposition of cells onto the portion except cell-trapping pores 7 of the surface of the cell collection plate 5, surface treatment is applied in this case. An optical system for confirming that cells are trapped is not integrated in this Example; however, such an optical system may be integrated.

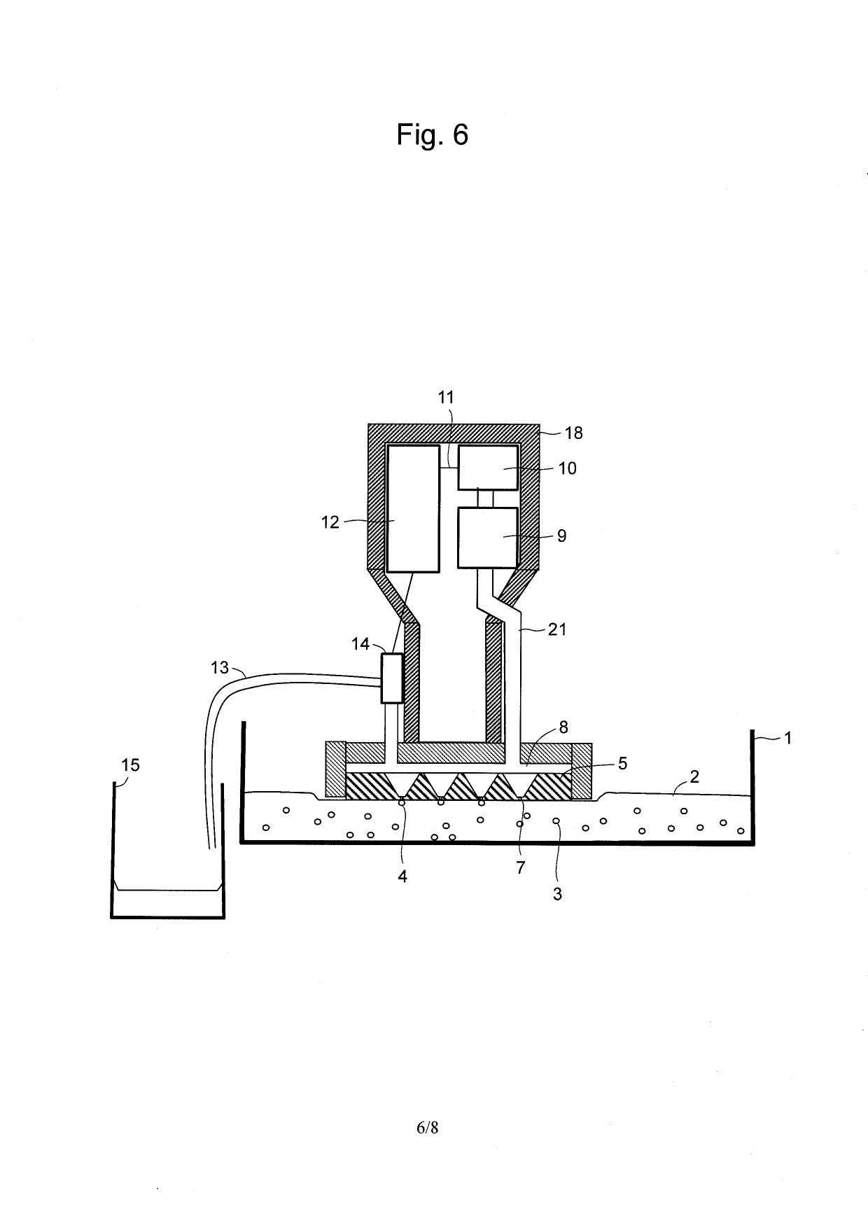

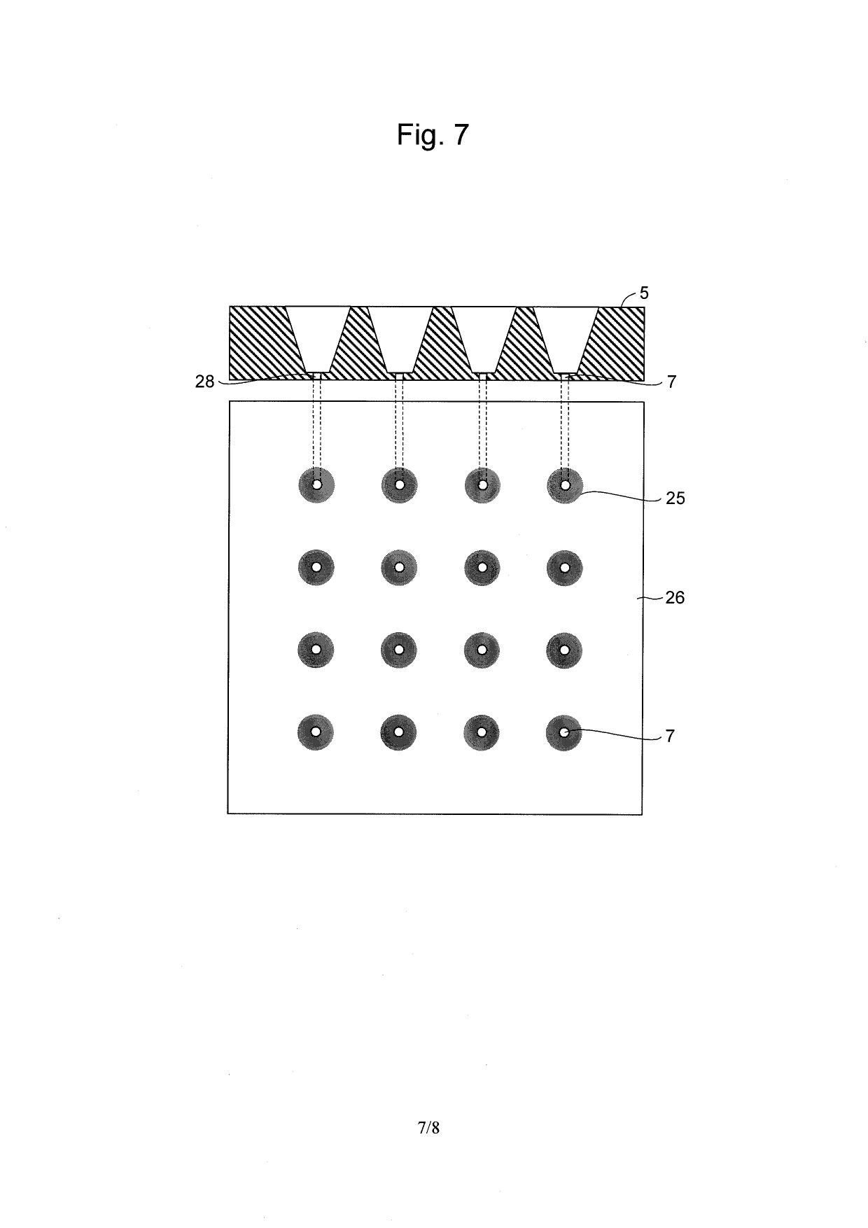

FIG. 6 shows the structure of the system in a step of trapping cells 3 suspending in a petri dish. Similarly to Example 1, the cell collection system is soaked such that a one-side surface is in contact with a PBS buffer 2 suspending cultured cells 3 in a petri dish 1 made of glass. In the cell collection plate 5, pores 7 are formed for collecting cells. The diameter of an opening portion in a cell collection surface herein is set at 4 μm. The pores 7 (16 pores) are arrayed at intervals of 9 mm, so as to correspond to the intervals of reaction vessels arrayed in a 96-well plate, in a reticular pattern of 4×4. FIG. 7 shows a sectional view of the cell collection plate 5 and a top view of the surface (cell collection surface) of the cell collection plate 5 to be contacted with cells. In this Example, the cell collection plate 5 is constituted of a single layer and the structural components except pores 7 were shaped by use of injection molding. Polyolefin was employed as the material; however, a resin such as polypropylene and polycarbonate may be used. Furthermore, a semiconductor may be used as the material and processed by use of a semiconductor processing technique. During injection molding, thin film portions 28 having a diameter 30μm and a thickness of 5 μm were formed and the center portion of each of the thin-film portions was punched with an excimer laser to form pores 7 having a diameter of 4 μm. The thickness of the cell collection plate 5 was 0.5 mm for maintaining the shape. The size of the cell collection plate was 45×45 mm to 500×500 mm and a thickness thereof was set at 1 mm.

Furthermore, FIG. 7 (top view) shows a surface treatment pattern of a cell collection surface. The cell collection pores 7 (16 pores) are arrayed at intervals of 9 mm in the form of a square lattice. The region 25 around a pore 7 having a diameter of 50 μm is hydrophilized; whereas, the other region 26 remains as it is since the surface of polyvinylidene chloride is water repellant. Hydrophilic treatment was performed for 10 minutes in accordance with a UV ozone treatment (UV irradiation (including light having a wavelength of 254 nm or 176 nm) under an oxygen atmosphere) by applying metal mask only the vicinities of pores 7 serving as opening portions. After resist patterning (usually used in a semiconductor process) is performed, a silane coupling agent having an OH group as a functional group may be reacted only with the opening portions to form the hydrophilic pattern. Herein, more simple treatment method was selected. Owing to this treatment, after a solution is suctioned from a petri dish and trapping of cells is confirmed and the system is pulled up from the petri dish, liquid drops virtually remain only in the hydrophilized portions and cells are also adsorbed only around pores.

Herein, the hydrophilic surface and water repellent surface will be defined. FIG. 8 shows sectional views of liquid drops of PBS buffer dripped on a hydrophilized portion 25 and a water repellent surface 26 (not hydrophilized). The water contact angle θ₁ of a liquid drop 81 dripped on hydrophilized surface 25 is smaller than water contact angle θ₂ of a liquid drop 82 dripped on the water repellent surface 26. In the present invention, the hydrophilic surface and water repellant surface are defined based on large or small of these two angles.

The inside of the suction chamber 8 is filled with PBS buffer 2 before cells 3 are trapped. This is realized by feeding a solution from a solution reservoir 9 (storing PBS buffer) by use of a pump 10 through a liquid-feeding tube 21. Furthermore, feeding (with application of pressure) of the buffer to the suction chamber 8, suctioning (by reducing pressure), and opening to the atmosphere are controlled by sending a control signal to the pump 10 through the signal line 11 by the controller 12.

Next, to trap cells 3, it is necessary to produce pressure difference between the cell collection surface and rear surface of pores 7 of the cell collection plate 5. For this, a discharge tube 13 was provided to an appropriate position lower than the liquid surface of a buffer in the petri dish 1 and a flow rate was controlled by a flow controller 14 provided in the middle of the discharge tube 13. Reference numeral 15 denotes a wastewater container, which collect discharged PBS.

In this case, the pressure of the inner portion of the suction chamber 8 is controlled by the controller 12 so as to keep an appropriate negative pressure (weak pressure enough to keep cells from destroying) relative to the exterior portion.

Next, trapped cells 4 are ejected to a reaction vessel plate in the same manner as in the Examples above.

Note that in this Example, sixteen pores 7 were arrayed in the cell collection plate 5; however, the number of pores may be 1, 96 or 384. In the case of a single cell, the possibility of introducing unnecessary cells into a reaction vessel can be reduced; whereas in the cases of 96 and 384 cells, throughput can be improved.

Note that the present invention is not limited to the aforementioned Examples and include variously modified examples. For example, the present invention is more specifically described by way of the aforementioned Examples in order to make the invention easily understood. The invention is not always limited to that having all constitutions. Furthermore, part of the constitutions of an Example may be replaced for a part of the constitutions of other Examples. Moreover, constitutions of other Examples may be added to the constitutions of an Example. Furthermore, with respect to a part of the constitution of each Example, addition, deletion or substitution of other constitutions can be made.

All publications, patents and patent applications cited in the specification are incorporated herein in their entirety as a reference.

### REFERENCE SIGNS LIST

1 Petri dish
2 PBS buffer
3 Cells
4 Trapped cells
5 Cell collection plate
6 Portion in contact with a cell collection surface
7 Pores
8 Suction chamber
9 Solution reservoir
10 Pump
11 Signal line
12 Controller
13 Discharge tube
14 Flow controller
15 Wastewater container
16 Aspheric surface lens
17 Fiber bundle
18 CMOS sensor
19 Optical module
20 Micrometer
21 Liquid-feeding tube
22 White LED for illumination
23 Signal line
25 Portion hydrophilized
26 Water repellent surface portion
28 Thin film portion
31 Reaction vessel plate
32 Reaction vessel
33 Aligning pin
34 Aligning hole
51 Semiconductor laser
52 Fiber bundle
53 Field lens
54 Dichroic mirror
55 Objective lens
56 Imaging lens
57 Band-pass filter
58 Cool CCD
59 Optical module
81 Liquid drop
82 Liquid drop

1. A cell collection system, comprising
a cell collection plate having at least one pore formed therein and one surface that can be soaked or in contact with a solution comprising a cell,
means for trapping a cell in the pore by suctioning the solution comprising a cell from the pore,
means for ejecting the cell trapped in the pore, and
means for obtaining an optical image of a vicinity of the pore in the cell collection plate.

2. The cell collection system according to claim 1, wherein a vicinity of the pore in the surface of the cell collection plate is hydrophilic and/or the other portion except the vicinity of the pore of the surface is water repellent.

3. A cell collection system, comprising
a cell collection plate having at least one pore formed therein and one surface that can be soaked or in contact with a solution comprising a cell,
means for trapping a cell in the pore by suctioning the solution comprising a cell from the pore, and
means for ejecting the cell trapped in the pore,
wherein
the vicinity of the pore in the surface of the cell collection plate is hydrophilic and/or the other portion except the vicinity of the pore is water repellent.

4. The cell collection system according to claim 1, wherein the pore is two-dimensionally or one-dimensionally arrayed in the cell collection plate.

5. The cell collection system according to claim 1, wherein the diameter of the pore is smaller than the diameter of the cell.

6. The cell collection system according to claim 1, wherein at least a part of the cell collection plate is transparent.

7. The cell collection system according to claim 1, wherein a periphery of the pore in the cell collection plate is transparent.

8. The cell collection system according to claim 1, wherein the means for obtaining an optical image of the vicinity of the pore in the cell collection plate comprises a light fiber bundle.

9. The cell collection system according to claim 1, wherein the cell collection plate in the vicinity of the pore have a shape projecting on the side of the surface to be soaked or in contact with the solution comprising a cell.

10. The cell collection system according to claim 1, further comprising illumination means.

11. The cell collection system according to claim 1, further comprising a computer and software for analyzing the optical image of the vicinity of the pore, wherein the cell collection system automatically recognizes the trapping of the cell in the pore based on contrast difference between an image of the pore and an image of the other portion except the pore.

12. The cell collection system according to claim 1, wherein the means for obtaining an optical image comprises a fluorescence excitation light source, an optical system for detecting fluorescence and an imaging device for obtaining a fluorescent image.

13. The cell collection system according to claim 1, further comprising a mechanism for washing the cell collection plate.

14. A cell collection and dispensing system comprising
the collection system according to claim 1, and
a reaction vessel plate having at least one reaction vessel to which a cell is to be dispensed,
wherein the pore is formed in the cell collection plate at an interval corresponding to an array interval of the reaction vessel in the reaction vessel plate.

15. The cell collection and dispensing system according to claim 14, wherein the number of the pore in the cell collection plate matches with the number of the reaction vessel in the reaction vessel plate.

16. The cell collection and dispensing system according to claim 14, wherein the cell collection plate and the reaction vessel plate have means for aligning the cell collection plate to the reaction vessel plate.

17. The cell collection and dispensing system according to claim 16, wherein the alignment means is provided such that the pore in the cell collection plate is positioned off the center of the corresponding reaction vessel of the reaction vessel plate.

18. The cell collection and dispensing system according to claim 14, wherein the cell collection plate is discretely provided from the solution comprising a cell and the reaction vessel plate and can be moved without limit.

19. The cell collection and dispensing system according to claim 14, further comprising means capable of taking the optical image focused within the reaction vessel when the cell trapped in the pore of the cell collection plate is dispensed to the reaction vessel plate.

20. The cell collection system according to claim 3, wherein the pore is two-dimensionally or one-dimensionally arrayed in the cell collection plate.

21. The cell collection system according to claim 3, wherein the diameter of the pore is smaller than the diameter of the cell.

22. The cell collection system according to claim 3, wherein at least a part of the cell collection plate is transparent.

23. The cell collection system according to claim 3, wherein a periphery of the pore in the cell collection plate is transparent.

24. The cell collection system according to claim 3, wherein the cell collection plate in the vicinity of the pore have a shape projecting on the side of the surface to be soaked or in contact with the solution comprising a cell.

25. The cell collection system according to claim 3, further comprising illumination means.

26. The cell collection system according to claim 3, further comprising a mechanism for washing the cell collection plate.

27. A cell collection and dispensing system comprising
the collection system according to claim 3, and
a reaction vessel plate having at least one reaction vessel to which a cell is to be dispensed,
wherein the pore is formed in the cell collection plate at an interval corresponding to an array interval of the reaction vessel in the reaction vessel plate.

28. The cell collection and dispensing system according to claim 27, wherein the number of the pore in the cell collection plate matches with the number of the reaction vessel in the reaction vessel plate.

29. The cell collection and dispensing system according to claim 27, wherein the cell collection plate and the reaction vessel plate have means for aligning the cell collection plate to the reaction vessel plate.

30. The cell collection and dispensing system according to claim 29, wherein the alignment means is provided such that the pore in the cell collection plate is positioned off the center of the corresponding reaction vessel of the reaction vessel plate.

31. The cell collection and dispensing system according to claim 27, wherein the cell collection plate is discretely provided from the solution comprising a cell and the reaction vessel plate and can be moved without limit.

32. The cell collection and dispensing system according to claim 27, further comprising means capable of taking the optical image focused within the reaction vessel when the cell trapped in the pore of the cell collection plate is dispensed to the reaction vessel plate.
